(19) United States
(12) Patent Application Publication
(10) Pub. No.: US 2023/0295553 A1
(43) Pub. Date: Sep. 21, 2023

(54) SUBSTANCE INTRODUCTION UNIT AND SUBSTANCE INTRODUCTION DEVICE

(71) Applicant: TERUMO KABUSHIKI KAISHA, Tokyo (JP)

(72) Inventors: Youichi HIGUCHI, Kanagawa (JP); Yuu NAKANO, Kanagawa (JP)

(21) Appl. No.: 18/081,576

(22) Filed: Dec. 14, 2022

(30) Foreign Application Priority Data
Feb. 28, 2022 (JP) 2022-030394

(51) Int. Cl. C12M 1/42; C12M 1/26; C12M 3/06; C12M 1/00

(52) U.S. Cl. CPC C12M 35/02; C12M 23/14; C12M 23/16; C12M 23/40; C12M 33/14

(57) ABSTRACT

A substance introduction unit, used for introduction of a substance into a cell by electroporation, includes an accommodation container configured to accommodate a cell suspension containing the cell and the substance; and a pair of electrodes which has electrode surfaces exposed to an internal space of the accommodation container and is configured to apply a voltage to the cell suspension accommodated in the accommodation container, the accommodation container including a cell-impermeable member configured to partition the internal space of the accommodation container.

### CROSS REFERENCE TO RELATED APPLICATIONS

The present application claims the benefit of and priority, under 35 U.S.C. § 119, to Japanese Application Serial No. 2022-030394, filed on Feb. 28, 2022, entitled “Substance Introduction Unit and Substance Introduction Device,” the entire disclosure of which is hereby incorporated herein by reference, in its entirety, for all that it teaches and for all purposes.

### BACKGROUND

The present disclosure relates to a substance introduction unit and a substance introduction device.

Conventionally, techniques of introducing substances into cells by electroporation are known. For example, PCT Patent Publication No. WO 2021/177185, entitled “Substance Introduction Device and Substance Introduction Method,” discloses an electroporation device capable of repeatedly executing electroporation in the same container.

### BRIEF SUMMARY

In general, in the electroporation, an optimum electroporation execution condition, such as a voltage value of a voltage to be applied or time for the application, is different depending on an environmental factor such as the amount of a cell suspension to be treated, the volume of a container in which the treatment is executed, or the distance between a pair of electrodes to which a voltage is applied. Therefore, for example, even in a case where electroporation execution conditions for a small amount of cells are established with a certain container, it is necessary to reset the execution conditions again with a different container when the electroporation is executed in the different container in order to increase the amount of the cell suspension to be treated.

In this regard, the device disclosed in PCT Patent Publication No. WO 2021/177185 can repeatedly execute the electroporation in the same container without enlarging a container for execution of the electroporation, so that the total amount of a cell suspension to be treated can be increased without changing electroporation execution conditions.

However, there is a demand for further improvement in usefulness of the techniques of introducing substances into cells by the electroporation. For example, it is required not to decrease the substance introduction efficiency in electroporation when the electroporation is repeatedly executed in the same container.

An object of the present disclosure that has been made in view of such circumstances is to provide a substance introduction unit and a substance introduction device that improve usefulness of a technique of introducing a substance into a cell by electroporation.

A substance introduction unit according to an embodiment of the present disclosure is a substance introduction unit which is used for introduction of a substance into a cell by electroporation, and includes: an accommodation container configured to accommodate a cell suspension containing the cell and the substance; and a pair of electrodes having electrode surfaces exposed to an internal space of the accommodation container and configured to apply a voltage to the cell suspension accommodated in the accommodation container, the accommodation container including a cell-impermeable member configured to partition the internal space of the accommodation container.

In the substance introduction unit according to the embodiment of the present disclosure, the cell-impermeable member is preferably located on an inner side of the electrode surfaces exposed to the internal space of the accommodation container.

In the substance introduction unit according to the embodiment of the present disclosure, the cell-impermeable member is preferably a porous membrane having cell impermeability.

In the substance introduction unit according to the embodiment of the present disclosure, the cell-impermeable member is preferably configured to partition the internal space of the accommodation container into an internal space (e.g., first internal space) where the electrode surfaces of the pair of electrodes are exposed and into another internal space (e.g., second internal space).

In the substance introduction unit according to the embodiment of the present disclosure, the cell-impermeable member preferably has a cylindrical shape extending in a longitudinal direction of the accommodation container.

In the substance introduction unit according to the embodiment of the present disclosure, the cell-impermeable member is preferably configured to partition the internal space of the accommodation container into an internal space where the electrode surface of an anode out of the pair of electrodes is exposed and another internal space.

In the substance introduction unit according to the embodiment of the present disclosure, the cell-impermeable member is preferably arranged in the accommodation container out of contact with the electrode surfaces.

A substance introduction device according to an embodiment of the present disclosure is a substance introduction device which introduces a substance into a cell by electroporation, and includes the above-described substance introduction unit.

The substance introduction unit and the substance introduction device according to the present disclosure can improve the usefulness of the technique of introducing the substance into the cell by the electroporation.

### BRIEF DESCRIPTION OF THE SEVERAL VIEWS OF THE DRAWINGS

FIG. 1 is a schematic diagram schematically illustrating a schematic configuration of a substance introduction device according to an embodiment of the present disclosure using a partial cross-sectional view;

FIG. 2 is a schematic cross-sectional view schematically illustrating a substance introduction unit illustrated in FIG. 1;

FIG. 3 is a schematic cross-sectional view schematically illustrating a modification of the substance introduction unit illustrated in FIG. 2; and

FIG. 4 is a flowchart illustrating an operation of the substance introduction device illustrated in FIG. 1.

### DETAILED DESCRIPTION

Hereinafter, a substance introduction device 1 according to an embodiment of the present disclosure will be described with reference to the drawings.

In the drawings, the same or equivalent portions are denoted by the same reference signs. In the description of the embodiment, the description regarding the same or equivalent portions is omitted or simplified as appropriate.

The substance introduction device 1 according to the embodiment of the present disclosure introduces a substance into a cell by electroporation. Electroporation is a method in which an electrical pulse or the like is applied to a cell suspension containing a cell and a substance to be introduced into the cell to perforate a cell membrane of the cell and introduce the substance into the cell. Electroporation is also referred to as an electroporation method or a process of electroporation. In the embodiment of the present disclosure, the cell suspension is obtained by suspending, for example, an animal cell or a cell such as Escherichia coli, and a substance such as a gene, deoxyribonucleic acid (DNA), ribonucleic acid (RNA), nucleic acid, protein, a low molecular weight compound, a lipid molecule, or a liposome in a buffer solution (buffer).

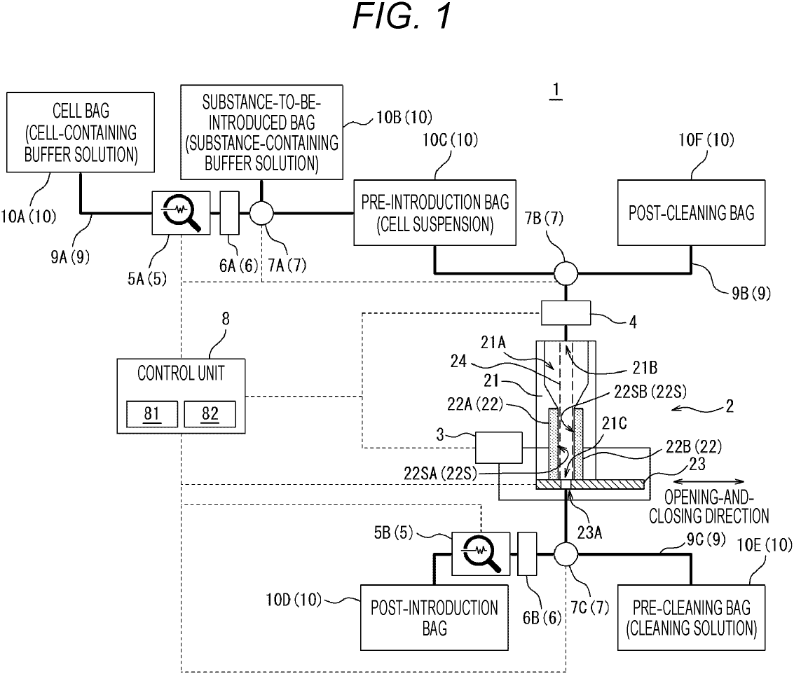

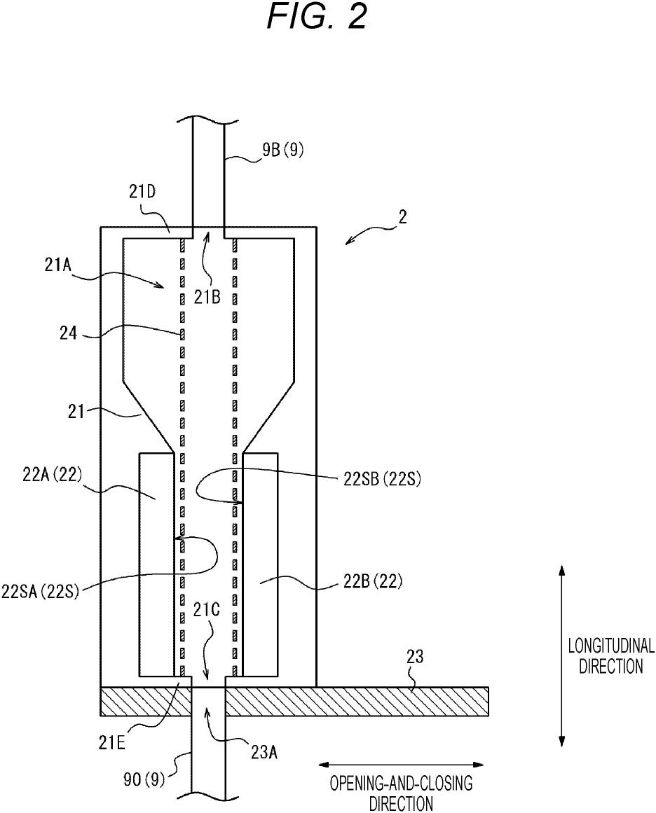

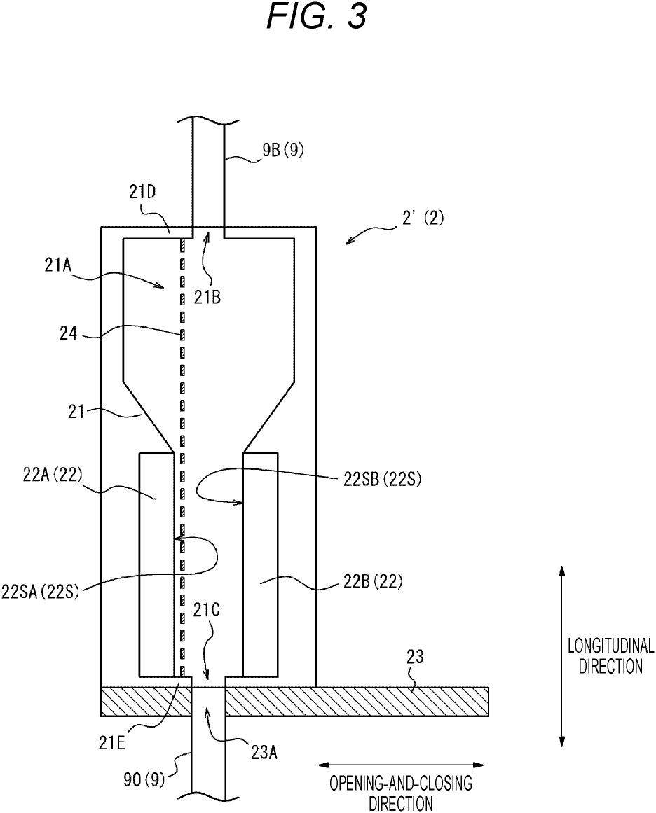

The substance introduction device 1 according to the embodiment of the present disclosure will be described with reference to FIGS. 1, 2, and 3. FIG. 1 is a schematic diagram schematically illustrating a schematic configuration of the substance introduction device 1 according to the embodiment of the present disclosure using a partial cross-sectional view. FIG. 2 is a schematic cross-sectional view schematically illustrating a substance introduction unit 2 illustrated in FIG. 1. FIG. 3 is a schematic cross-sectional view schematically illustrating a modification (a substance introduction unit 2′) of the substance introduction unit 2 illustrated in FIG. 2. In FIGS. 1 to 3, the substance introduction unit 2 is illustrated in a cross-sectional view. As illustrated in FIG. 1, the substance introduction device 1 includes the substance introduction unit 2, a voltage application unit 3, a liquid delivery unit 4, a cell counter 5, a filtering unit 6, a valve unit 7, and a control unit 8.

As will be described later, a plurality of bags 10 are attached to the substance introduction device 1 via liquid delivery paths 9 such as liquid delivery tubes. In the embodiment, a cell bag 10A, a substance-to-be-introduced bag 10B, a pre-introduction bag 10C, a post-introduction bag 10D, a pre-cleaning bag 10E, and a post-cleaning bag 10F are attached to the substance introduction device 1 as illustrated in FIG. 1. The cell bag 10A accommodates cells together with a buffer solution. The substance-to-be-introduced bag 10B accommodates a substance to be introduced into the cells together with a buffer solution. The pre-introduction bag 10C accommodates a cell suspension before substance introduction by electroporation. The post-introduction bag 10D accommodates a cell suspension into which the substance has been introduced by electroporation. The pre-cleaning bag 10E accommodates a cleaning solution to be used for cleaning of the substance introduction unit 2. The post-cleaning bag 10F accommodates the cleaning solution after cleaning the substance introduction unit 2.

As illustrated in FIG. 1, the cell bag 10A and the substance-to-be-introduced bag 10B are connected to the pre-introduction bag 10C by a liquid delivery path 9A in the substance introduction device 1. The pre-introduction bag 10C and the post-cleaning bag 10F are connected to a first opening 21B of the substance introduction unit 2 by a liquid delivery path 9B. The post-introduction bag 10D and the pre-cleaning bag 10E are connected to a second opening 21C of the substance introduction unit 2 by a liquid delivery path 9C.

In the embodiment, the inside of the substance introduction unit 2 is cleaned by a cleaning solution after execution of the electroporation process. This cleaning removes stains, such as cell residues or air bubbles, adhering to the inside of the substance introduction unit 2, particularly, electrode surfaces 22S of a pair of electrodes 22. The cell residues include, for example, a dead cell, a damaged cell (fracture), protein, nucleic acid, or the like. Since the inside of the substance introduction unit 2 is cleaned after execution of the electroporation process as described above, the substance introduction efficiency in the electroporation is less likely to decrease when the electroporation is repeatedly executed in the same container. The cleaning solution is, for example, a buffer solution, but is not limited thereto.

The substance introduction unit 2 is an instrument that is used to introduce the substance into the cell by electroporation. As illustrated in FIG. 2, the substance introduction unit 2 includes an accommodation container 21, the pair of electrodes 22, and an opening and closing unit 23.

The accommodation container 21 is configured to accommodate the cell suspension containing the cell and the substance to be introduced into the cell. The accommodation container 21 is, for example, a cuvette. The accommodation container 21 may be made of, for example, glass, plastic, silicon, quartz, or the like. The accommodation container 21 internally partitions an internal space 21A that is long and can accommodate the cell suspension. A size of the internal space 21A may be determined depending on the amount of the cell suspension treated in one electroporation, and is, for example, 10 microliters to 10 milliliters, desirably 40 microliters to 400 microliters.

The accommodation container 21 is a cylindrical container having the first opening 21B and the second opening 21C. The internal spaces 21A partitioned inside the accommodation container 21 communicate with the outside of the accommodation container 21 through the first opening 21B and the second opening 21C, respectively. In the embodiment, the accommodation container 21 includes a first flat plate portion 21D having the first opening 21B on one end side of the accommodation container 21 in a longitudinal direction, and includes a second flat plate portion 21E having the second opening 21C on the other end side opposite to the first opening 21B of the accommodation container 21 in the longitudinal direction. Thus, the accommodation container 21 is configured such that a liquid that has been charged into the internal space 21A through one of the first opening 21B and the second opening 21C can be discharged to the outside through the other of the first opening 21B and the second opening 21C.

The pair of electrodes 22 is configured to apply a voltage to the cell suspension accommodated in the accommodation container 21. In the embodiment, the pair of electrodes 22 includes a first electrode 22A and a second electrode 22B. Both the first electrode 22A and the second electrode 22B are plate-shaped electrodes and are made of an energizable (e.g., capable of being electrically energized) material such as metal or a conductive polymer. In the embodiment, the first electrode 22A is assumed to be an anode, and the second electrode 22B is assumed to be a cathode. The first electrode 22A and the second electrode 22B are attached to the accommodation container 21 so as to face each other with the internal space 21A of the accommodation container 21 sandwiched therebetween. The pair of electrodes 22 generates an electrical pulse in the internal space 21A of the accommodation container 21 sandwiched between the pair of electrodes 22 when power is applied from the voltage application unit 3 in a state where the cell suspension is accommodated in the accommodation container 21. Thus, the electroporation is executed in the accommodation container 21 so that the substance is introduced into the cell contained in the cell suspension. A distance between the electrodes constituting the pair of electrodes 22 is set to a distance suitable for executing the electroporation, and is, for example, 0.1 mm to 1 cm, and is desirably in the range of 1 mm to 5 mm.

The pair of electrodes 22 is provided on a side surface of the accommodation container 21 sandwiched between the first opening 21B and the second opening 21C in the longitudinal direction of the accommodation container 21. The pair of electrodes 22 has the electrode surfaces 22S exposed to the internal space 21A of the accommodation container 21. Specifically, the first electrode 22A is attached to the accommodation container 21 such that the electrode surface 22SA is exposed to the internal space 21A of the accommodation container 21. The second electrode 22B is attached to the accommodation container 21 such that the electrode surface 22SB is exposed to the internal space 21A of the accommodation container 21. In the present disclosure, the electrode surface 22SA of the first electrode 22A and the electrode surface 22SB of the second electrode 22B are collectively referred to simply as the “electrode surfaces 22S” when not particularly distinguished.

The electrode surfaces 22S of the pair of electrodes 22 exposed to the internal space 21A of the accommodation container 21 may be subjected to polymer coating. The polymer coating is, for example, polyethylene glycol (PEG) coating. However, the coating is not limited to the PEG coating, and may be a non-cell-adhesive polymer coating, superhydrophilic polymer coating, or any coating having cell non-adhesion or superhydrophilicity. Thus, the stains, such as the cell residues or air bubbles, are less likely to adhere to the electrode surfaces 22S of the pair of electrodes 22 exposed to the internal space 21A of the accommodation container 21, and the substance introduction efficiency in the electroporation is less likely to decrease.

The pair of electrodes 22 may be configured to be detachable from the accommodation container 21. Alternatively, the accommodation container 21 and the pair of electrodes 22 may be integrated as cuvette electrodes.

The opening and closing unit 23 is configured to be capable of opening and closing the opening of the accommodation container 21. In the embodiment, the opening and closing unit 23 is attached to the accommodation container 21 so as to cover an end portion of the accommodation container 21 on the second opening 21C side. Thus, the opening and closing unit 23 is configured to be capable of opening and closing the second opening 21C of the accommodation container 21. For example, as illustrated in FIG. 2, the opening and closing unit 23 is a plate-shaped member that has a through-hole 23A and is slidable in an opening-and-closing direction relative to the accommodation container 21. The liquid delivery path 9C may be attached to the through-hole 23A of the opening and closing unit 23 by fitting or the like. The opening and closing unit 23 is slid (e.g., slidably moved) to align a position of the through-hole 23A with a position of the second opening 21C of the accommodation container 21, whereby the opening and closing unit 23 opens the second opening 21C of the accommodation container 21. The opening and closing unit 23 is slid (e.g., slidably moved) to cause the position of the through-hole 23A to be misaligned (e.g., arranged out of alignment) from the position of the second opening 21C of the accommodation container 21, whereby the opening and closing unit 23 closes the second opening 21C of the accommodation container 21.

In the accommodation container 21 of the substance introduction unit 2, the first opening 21B may be configured to be located vertically above the second opening 21C. Thus, when the movement of the cell suspension by gravity is utilized, the cell suspension can be accommodated in the internal space 21A of the accommodation container 21 or can be discharged from the internal space 21A by opening or closing the second opening 21C.

In the embodiment, the opening and closing unit 23 is configured to be able to automatically open and close the opening of the accommodation container 21 under the control of the control unit 8. For example, the opening and closing unit 23 may include a motor for sliding the opening and closing unit 23 itself relative to the accommodation container 21. However, the opening and closing unit 23 may be configured to be able to manually open and close the opening of the accommodation container 21. Further, the opening and closing unit 23 is not limited to the above-described configuration, and may be any instrument capable of automatically or manually opening and closing the opening of the accommodation container 21, such as an opening and closing valve, a lid, or a door. The substance introduction unit 2 may further include an opening and closing unit 23 configured to be capable of opening and closing the first opening 21B, in addition to the opening and closing unit 23 configured to be capable of opening and closing the second opening 21C of the accommodation container 21.

In the substance introduction unit 2 according to the embodiment, the accommodation container 21 includes a cell-impermeable member 24 configured to partition the internal space 21A of the accommodation container 21. The cell-impermeable member 24 is, for example, a porous membrane having cell impermeability. A pore diameter of the porous membrane is smaller than a diameter of the cell to be electroporated. The pore diameter of the porous membrane is, for example, 5 µm to 50 µm, but is not limited thereto, and may be determined in accordance with a cell to be electroporated. Further, the cell-impermeable member 24 is desirably made of a material, such as carbon or polymer, having electrical conduction. However, a structure and a material of the cell-impermeable member 24 are not particularly limited as long as the cell-impermeable member 24 is a member that does not allow permeation of cells. As described above, the internal space 21A of the accommodation container 21 is partitioned into a plurality of spaces by the cell-impermeable member 24 so that a space in which the cells are movable in the internal space 21A of the accommodation container 21. Thus, it is possible to limit a range that may be contaminated due to adhesion of cells including a dead cell, a damaged cell, or the like in the substance introduction unit 2, and the substance introduction efficiency in the electroporation is less likely to decrease when the electroporation is repeatedly executed in the same container.

The cell-impermeable member 24 is located on the inner side of the electrode surfaces 22S of the pair of electrodes 22 exposed to the internal space 21A of the accommodation container 21 in the internal space 21A of the accommodation container 21. Thus, the cell-impermeable member 24 is configured to partition the internal space 21A of the accommodation container 21 into an internal space where the electrode surfaces 22S of the pair of electrodes 22 are exposed and the other internal space. In the embodiment, the other internal space of the internal space 21A of the accommodation container 21 communicates with the outside of the accommodation container 21 through the first opening 21B and the second opening 21C. When the accommodation container 21 is filled with the cell suspension such that the cells move only in the other internal space out of the internal space 21A of the accommodation container 21, cells including a dead cell, a damaged cell, or the like are less likely to adhere to the electrode surfaces 22S of the pair of electrodes 22, and the substance introduction efficiency in the electroporation is less likely to decrease.

In the embodiment, the cell-impermeable member 24 has a cylindrical shape extending in the longitudinal direction of the accommodation container 21, and has both ends fixed to the first flat plate portion 21D and the second flat plate portion 21E so as to surround the first opening 21B and the second opening 21C, respectively, as illustrated in FIG. 2. Thus, cells including a dead cell, a damaged cell, or the like are less likely to adhere to the electrode surfaces 22S of the pair of electrodes 22 located on the outer side of the cylindrical cell-impermeable member 24 and the side surface of the accommodation container 21, and the substance introduction efficiency in the electroporation is less likely to decrease.

However, the shape of the cell-impermeable member 24 is not limited to the cylindrical shape. For example, the cell-impermeable member 24 may have a shape including two plates. In such a case, the cell-impermeable member 24 may be arranged in the accommodation container 21 so as to partition the internal space 21A of the accommodation container 21 into an internal space where the electrode surface 22SA of the first electrode 22A is exposed, an internal space communicating with the outside of the accommodation container 21 through the first opening 21B and the second opening 21C, and an internal space where the electrode surface 22SB of the second electrode 22B is exposed.

Furthermore, the cell-impermeable member 24 may be configured to partition the internal space 21A of the accommodation container 21 into an internal space where the electrode surface 22SA of the first electrode 22A, which is the anode, out of the pair of electrodes 22 is exposed and the other internal space. Specifically, as illustrated as the substance introduction unit 2′ in FIG. 3, the cell-impermeable member 24 may be formed in a single plate shape and arranged in the accommodation container 21 so as to partition the internal space 21A of the accommodation container 21 into an internal space where the electrode surface 22SA of the first electrode 22A, which is the anode, out of the pair of electrodes 22 is exposed and an internal space communicating with the outside of the accommodation container 21 through the first opening 21B and the second opening 21C. Thus, the cell-impermeable member 24 covers only the electrode surface 22SA of the anode to which cells including a dead cell, a damaged cell, or the like are likely to adhere when a voltage is applied between the pair of electrodes 22, so that it is possible to suppress an increase in materials constituting the substance introduction unit 2′ and to suppress an increase in manufacturing cost of the substance introduction unit 2′.

Referring again to FIG. 2, the cell-impermeable member 24 is arranged in the accommodation container 21 out of contact with the electrode surfaces 22S of the pair of electrodes 22. Thus, the cell-impermeable member 24 and cells adhering to the cell-impermeable member 24 come into contact with the electrode surfaces 22S of the pair of electrodes 22, and the stability and uniformity of the pulse generated by the pair of electrodes 22 are hardly impaired. However, the cell-impermeable member 24 may be arranged in the accommodation container 21 to be in contact with the electrode surfaces 22S of the pair of electrodes 22.

The cell-impermeable member 24 may be configured to be detachable from the accommodation container 21. Thus, the accommodation container 21 can be continuously used by replacing only the cell-impermeable member 24 when the cell-impermeable member 24 is contaminated due to adhesion of cells such as dead cells or damaged cells. However, the cell-impermeable member 24 may be integrated with the accommodation container 21.

The substance introduction unit 2 is detachable from the substance introduction device 1. Thus, the substance introduction unit 2 can be replaced with a new substance introduction unit 2 in the case of being damaged, degraded, or the like. Alternatively, the substance introduction units 2 respectively having different volumes of the accommodation containers 21 can be attached to the substance introduction device 1 in accordance with the amount of the cell suspension to be treated in one electroporation. However, the substance introduction unit 2 may be integrated with the substance introduction device 1 or is not necessarily detachable from the substance introduction device 1.

Referring again to FIG. 1, the voltage application unit 3 is an instrument that applies a voltage between the pair of electrodes 22. The voltage application unit 3 is not particularly limited as long as a voltage can be applied between the pair of electrodes 22. The voltage application unit 3 may include, for example, a storage battery or a dry battery. Further, for example, the voltage application unit 3 may include an adapter or the like for receiving power supply from an external power source to receive the power supply from the external power source. The voltage application unit 3 is connected to each of the pair of electrodes 22 by a conductive wire or the like so as to be capable of supplying power as indicated by a solid line in FIG. 1. Thus, the voltage application unit 3 applies a predetermined voltage between the pair of electrodes 22, so that the electroporation is executed in the accommodation container 21, and the substance is introduced into the cells contained in the cell suspension. In the embodiment, it is assumed that the first electrode 22A serves as the anode and the second electrode 22B serves as the cathode when the voltage is applied by the voltage application unit 3.

The voltage application unit 3 may include one or more sensors. The sensor is, for example, an ohmmeter, a voltmeter, an ammeter, or the like. Thus, the voltage application unit 3 can measure a resistance value, a voltage value, or a current value between the pair of electrodes 22 by the sensor, and adjust the voltage applied between the pair of electrodes 22.

The liquid delivery unit 4 includes one or more liquid delivery pumps. The liquid delivery pump is, for example, a syringe-type, suction-type, or peristaltic pump, but is not limited thereto. The liquid delivery unit 4 is configured to be automatically operable under the control of the control unit 8, but may be configured to be manually operable. In the embodiment, the liquid delivery unit 4 is provided in the liquid delivery path 9B connected to the first opening 21B of the accommodation container 21 of the substance introduction unit 2. Thus, the liquid delivery unit 4 can deliver a liquid, such as the cell suspension or the cleaning solution, to the internal space 21A of the accommodation container 21. However, the liquid delivery unit 4 may be provided at any position of the liquid delivery path 9 included in the substance introduction device 1.

The liquid delivery unit 4 fills the accommodation container 21 with the cell suspension before voltage application through the first opening 21B of the accommodation container 21, and discharges the cell suspension after voltage application from the accommodation container 21 through the second opening 21C. Further, the liquid delivery unit 4 fills the accommodation container 21 with the cleaning solution through the second opening 21C of the accommodation container 21, and discharges the cleaning solution from the accommodation container 21 through the first opening 21B. As described above, a direction in which the cleaning solution flows inside the accommodation container 21 of the substance introduction unit 2 is set to be opposite to a direction in which the cell suspension flows in the accommodation container 21 of the substance introduction unit 2, so that the stains, such as the cell residues or air bubbles, are likely to be peeled off from the accommodation container 21, and an effect of cleaning the pair of electrodes 22 and the accommodation container 21 and the pair of electrodes 22 can be improved. In particular, in a case where the substance introduction unit 2 is arranged such that the first opening 21B is higher than the second opening 21C, the cell residues or the like are likely to adhere to the lower side (that is, the second opening 21C side in the embodiment) of the accommodation container 21 by gravity, and thus, it is effective to cause the cleaning solution to flow from the second opening 21C side.

The cell counter 5 is configured to count living cells. The living cell is a cell which is not dead or damaged and into which the substance can be introduced by electroporation. The cell counter 5 includes, for example, equipment such as a flow cytometer, a particle size distribution meter, and a micropore device. In the embodiment, the substance introduction device 1 includes a cell counter 5A provided in the liquid delivery path 9A and a cell counter 5B provided in the liquid delivery path 9C. However, the cell counter 5 may be provided at any position of the liquid delivery path 9 included in the substance introduction device 1.

The cell counter 5A is configured to count living cells in the cell suspension before voltage application with which the accommodation container 21 is to be filled. Thus, quantities or ratios of the cells contained in the cell suspension and the substance to be introduced into the cells can be appropriately set by adjusting the quantity of the substance to be mixed in the cell suspension in accordance with the number of the living cells, and the substance introduction efficiency in the electroporation can be improved.

The cell counter 5B is configured to count living cells in the cell suspension after voltage application discharged from the accommodation container 21. Thus, it is possible to grasp the number of living cells into which the substance has been introduced without dying or being damaged by electroporation. Furthermore, the number of the cell residues remaining in the accommodation container 21 of the substance introduction unit 2 can be estimated based on the number of the living cells.

The filtering unit 6 is configured to remove the cell residues. As described above, the cell residues include the dead or damaged cell, the protein, the nucleic acid, or the like. The filtering unit 6 includes, for example, magnetic beads, nonwoven fabric, an electret filter (electrostatic filter material), or the like. In the embodiment, the substance introduction device 1 includes a filtering unit 6A provided in the liquid delivery path 9A and a filtering unit 6B provided in the liquid delivery path 9C. However, the filtering unit 6 may be provided at any position of the liquid delivery path 9 included in the substance introduction device 1.

The filtering unit 6A is configured to remove cell residues from the cell suspension before voltage application with which the accommodation container 21 is to be filled. Thus, the cell residues contained in the cell suspension are reduced, and the substance introduction efficiency in the electroporation can be improved. Further, since the cell residues are removed in advance from the cell suspension with which the accommodation container 21 of the substance introduction unit 2 is to be filled, the accommodation container 21 and the pair of electrodes 22 are less likely to be contaminated, and the substance introduction efficiency in the electroporation can be further improved.

The filtering unit 6B is configured to remove cell residues from the cell suspension after voltage application discharged from the accommodation container 21. This makes it easy to recover the living cell into which the substance has been introduced by the electroporation.

The valve unit 7 is configured to be capable of opening and closing the liquid delivery path 9 or capable of switching a flow path of the liquid delivery path 9. The valve unit 7 includes, for example, an electromagnetic valve and the like, and is configured to be automatically operable under the control of the control unit 8. However, the valve unit 7 may be configured to be manually operable.

The control unit 8 includes a memory 81 and a processor 82. As the control unit 8, for example, a computer, such as an electroporator, a personal computer, a smartphone, or a tablet terminal, may be used.

The memory 81 is, for example, a semiconductor memory, a magnetic memory, an optical memory, or the like. The memory 81 functions as, for example, a main storage device, an auxiliary storage device, or a cache memory. The memory 81 stores any information to be used for an operation of the substance introduction device 1. For example, the memory 81 stores a system program, an application program, embedded software, or the like.

The processor 82 may be a general-purpose processor, such as a central processing unit (CPU), or a dedicated processor specialized for specific processing. The processor 82 may include, for example, a dedicated circuit such as a field-programmable gate array (FPGA) or an application-specific integrated circuit (ASIC).

As indicated by a broken line in FIG. 1, the control unit 8 is connected to each of the components of the substance introduction device 1 such as the opening and closing unit 23, the voltage application unit 3, the liquid delivery unit 4, the cell counter 5, and the valve unit 7 of the substance introduction unit 2 so as to be capable of communicating therewith in a wired or wireless manner. As a result, the control unit 8 controls these components in order to implement functions of the substance introduction device 1.

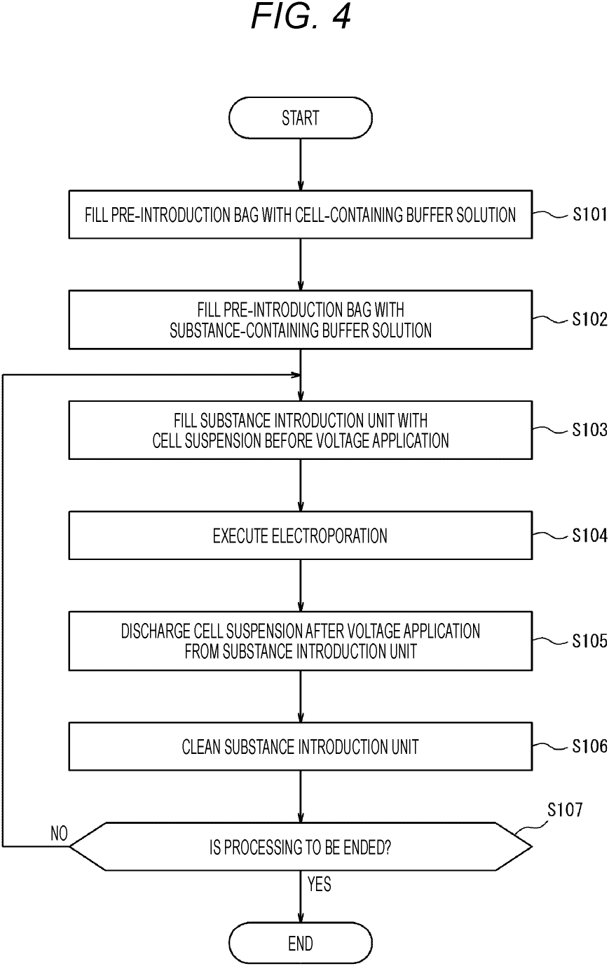

The operation of the substance introduction device 1 according to the embodiment of the present disclosure will be described with reference to FIG. 4. FIG. 4 is a flowchart illustrating the operation of the substance introduction device 1 illustrated in FIG. 1. This operation corresponds to a substance introduction method that is executed by the substance introduction device 1 according to the embodiment. In execution of the operation, the above-described substance introduction unit 2 is prepared in advance.

In steps S101 and S102, the control unit 8 prepares the cell suspension before substance introduction by electroporation.

Step S101: The control unit 8 fills the pre-introduction bag 10C with the cell-containing buffer solution from the cell bag 10A.

In the embodiment, the control unit 8 controls the valve unit 7A to fill the pre-introduction bag 10C with a predetermined amount of the cell-containing buffer solution from the cell bag 10A via the liquid delivery path 9A. At this time, since the filtering unit 6A is provided in the liquid delivery path 9A, the filtering unit 6A removes cell residues from the cell suspension before voltage application. Further, the control unit 8 controls the cell counter 5A to count the number of living cells present in the cell-containing buffer solution at the time of filling the pre-introduction bag 10C with the cell-containing buffer solution. The control unit 8 may adjust the amount of the cell-containing buffer solution to be charged into the pre-introduction bag 10C from the cell bag 10A in accordance with the number of the living cells counted by the cell counter 5A.

Step S102: The control unit 8 fills the pre-introduction bag 10C with the substance-containing buffer solution from the substance-to-be-introduced bag 10B.

In the embodiment, the control unit 8 controls the valve unit 7A to fill the pre-introduction bag 10C with the substance-containing buffer solution from the substance-to-be-introduced bag 10B via the liquid delivery path 9A. At this time, the control unit 8 adjusts the amount of the substance-containing buffer solution to be charged into the pre-introduction bag 10C from the substance-to-be-introduced bag 10B in accordance with the number of the living cells counted in step S101. However, the control unit 8 may fill the pre-introduction bag 10C with a predetermined amount of the substance-containing buffer solution from the substance-to-be-introduced bag 10B.

Through steps S101 and S102, the cell-containing buffer solution and the substance-containing buffer solution are suspended in the pre-introduction bag 10C, and the cell suspension before substance introduction by electroporation is prepared. However, the processing in steps S101 and S102 may be omitted by preparing the pre-introduction bag 10C in which the cell suspension before substance introduction by electroporation is accommodated in advance.

In steps S103 to S105, the control unit 8 introduces the substance into the cells by the electroporation.

Step S103: The control unit 8 fills the substance introduction unit 2 with the cell suspension before voltage application.

Specifically, the control unit 8 controls the valve unit 7B to connect the pre-introduction bag 10C and the first opening 21B of the accommodation container 21 by the liquid delivery path 9B. Then, the control unit 8 controls the opening and closing unit 23 of the substance introduction unit 2 to close the second opening 21C of the accommodation container 21. Thereafter, the control unit 8 controls the liquid delivery unit 4 to fill the accommodation container 21 with the cell suspension before voltage application from the pre-introduction bag 10 through the first opening 21B of the accommodation container 21. At this time, the control unit 8 may control the liquid delivery unit 4 so as to fill the accommodation container 21 with a predetermined amount of the cell suspension.

At this time, since the cell-impermeable member 24 is provided in the accommodation container 21, cells contained in the cell suspension are less likely to adhere to the electrode surfaces 22S of the pair of electrodes 22.

Step S104: The control unit 8 executes the electroporation.

Specifically, the control unit 8 controls the voltage application unit 3 to apply power to the pair of electrodes 22 to generate an electrical pulse in the internal space 21A of the accommodation container 21. Thus, the control unit 8 applies a voltage to the cell suspension accommodated in the accommodation container 21 by the pair of electrodes 22.

Step S105: The control unit 8 discharges the cell suspension after voltage application from the substance introduction unit 2.

Specifically, the control unit 8 controls the valve unit 7C to connect the second opening 21C of the accommodation container 21 and the post-introduction bag 10D by the liquid delivery path 9C. Then, the control unit 8 controls the opening and closing unit 23 of the substance introduction unit 2 to open the second opening 21C of the accommodation container 21. Thereafter, the control unit 8 controls the liquid delivery unit 4 to discharge the cell suspension after voltage application from the accommodation container 21 to the post-introduction bag 10D through the second opening 21C of the accommodation container 21. However, the control unit 8 may discharge the cell suspension after voltage application from the accommodation container 21 to the post-introduction bag 10D through the second opening 21C of the accommodation container 21 by its own weight without controlling the liquid delivery unit 4 from the viewpoint of energy saving.

At this time, since the filtering unit 6B is provided in the liquid delivery path 9C, the filtering unit 6B removes cell residues from the cell suspension after voltage application. Further, the control unit 8 controls the cell counter 5B to count the number of living cells present in the cell suspension after voltage application at the time of discharging the cell suspension after voltage application to the post-introduction bag 10D.

Step S106: The control unit 8 executes cleaning of the substance introduction unit 2.

Specifically, the control unit 8 controls the valve unit 7B to connect the post-cleaning bag 10F and the first opening 21B of the accommodation container 21 by the liquid delivery path 9B. Then, the control unit 8 controls the valve unit 7C to connect the pre-cleaning bag 10E and the second opening 21C of the accommodation container 21 by the liquid delivery path 9C. Further, the control unit 8 controls the opening and closing unit 23 of the substance introduction unit 2 to open the second opening 21C of the accommodation container 21.

Thereafter, the control unit 8 controls the liquid delivery unit 4 to fill the accommodation container 21 with the cleaning solution from the pre-cleaning bag 10E through the second opening 21C of the accommodation container 21. Then, the control unit 8 controls the liquid delivery unit 4 to discharge the cleaning solution from the accommodation container 21 to the post-cleaning bag 10F through the first opening 21B. At this time, the control unit 8 may control the liquid delivery unit 4 so as to fill the accommodation container 21 with a predetermined amount of the cleaning solution.

Step S107: The control unit 8 determines whether or not to end the present processing.

Specifically, the control unit 8 determines whether or not the present processing has been repeated a predetermined number of times. The predetermined number of times may be stored in the memory 81 in advance. When it is determined that the processing has not been repeated the predetermined number of times (“NO” in step S107), the control unit 8 repeatedly executes the processing from step S103. On the other hand, when it is determined that the present processing has been repeated the predetermined number of times (“YES” in step S107), the control unit 8 ends the present processing. The control unit 8 may determine whether or not a total amount of the cell suspension subjected to the present processing has reached a predetermined amount instead of determining whether or not the present processing has been repeated the predetermined number of times.

As described above, the substance introduction unit 2 according to the present disclosure is the substance introduction unit 2 which is used for introduction of a substance into a cell by electroporation, and includes: the accommodation container 21 configured to contain a cell suspension containing the cell and the substance; and the pair of electrodes 22 having the electrode surfaces 22S exposed to the internal space 21A of the accommodation container 21 and configured to apply a voltage to the cell suspension accommodated in the accommodation container 21, the accommodation container 21 including the cell-impermeable member 24 configured to partition the internal space 21A of the accommodation container 21.

According to the substance introduction unit 2 having such a configuration, the electroporation can be repeatedly executed in the same container without enlarging the container for execution of the electroporation, so that a total amount of the cell suspension to be treated can be increased without changing an electroporation execution condition. Then, it is possible to limit the range that may be contaminated due to the adhesion of the cells including the dead cell, the damaged cell, or the like by partitioning the internal space 21A of the accommodation container 21 by the cell-impermeable member 24. Therefore, according to the substance introduction unit 2 of the present disclosure, the substance introduction efficiency in the electroporation is less likely to decrease when the electroporation is repeatedly executed in the same container, and the usefulness of a technique of introducing the substance into the cell by the electroporation.

Although the present disclosure has been described with reference to the drawings and embodiment, it should be noted that those skilled in the art can make various modifications and corrections on the basis of the present disclosure. Therefore, it should be noted that these modifications and corrections fall within the scope of the present disclosure. For example, the function or the like included in each means, each step, or the like can be rearranged not to be logically inconsistent, and a plurality of means, steps, or the like can be combined into one or divided.

For example, it has been described that the cell-containing buffer solution and the substance-containing buffer solution are suspended in the pre-introduction bag 10C in the above-described embodiment. However, the cell-containing buffer solution and the substance-containing buffer solution may be suspended when flowing into the pre-introduction bag 10C in the liquid delivery path 9A. This makes it possible to efficiently suspend the cell-containing buffer solution and the substance-containing buffer solution.

Further, for example, it has been described that the electroporation and the cleaning of the substance introduction unit 2 are alternately executed the same number of times in the above-described embodiment. However, the number of times of the electroporation may be different from the number of times of the cleaning. For example, the cleaning may be executed once while the electroporation is repeated a plurality of times in accordance with the likelihood of generation of the cell residues in the electroporation. Thus, it is possible to optimize the substance introduction efficiency in the electroporation and the processing speed of the electroporation.

Further, for example, it has been described that the entire operation (substance introduction method) of the substance introduction device 1 is automatically executed under the control of the control unit 8 in the above-described embodiment. However, a part or whole of the operation of the substance introduction device 1 may be manually executed. For example, some or all of the opening and closing of the opening and closing unit 23 of the substance introduction unit 2, the voltage application with respect to the pair of electrodes 22 by the voltage application unit 3, the delivery by the liquid delivery unit 4, the counting of living cells by the cell counter 5, the switching of the flow path of the liquid delivery path 9 by the valve unit 7, and the like may be manually executed. Thus, a structure of the substance introduction device 1 can be simplified, and an increase in manufacturing cost of the substance introduction device 1 can be suppressed.

Further, for example, an embodiment in which a general-purpose computer is configured to function as the control unit 8 according to the above-described embodiment is also possible. Specifically, a program describing processing contents for implementing the respective functions of the control unit 8 according to the above-described embodiment is stored in a memory of the general-purpose computer, and the program is read and executed by a processor. Therefore, the present disclosure can also be implemented as the program executable by the processor or a non-transitory computer-readable medium that stores the program.

The present disclosure relates to a substance introduction unit and a substance introduction device.

What is claimed is:

1. A substance introduction unit, which is used for introduction of a substance into a cell by electroporation, comprising:
an accommodation container configured to accommodate a cell suspension containing the cell and the substance; and
a pair of electrodes having electrode surfaces exposed to an internal space of the accommodation container and configured to apply a voltage to the cell suspension accommodated in the accommodation container,
wherein the accommodation container includes a cell-impermeable member configured to partition the internal space of the accommodation container.

2. The substance introduction unit according to claim 1, wherein the cell-impermeable member is located on an inner side of the electrode surfaces exposed to the internal space of the accommodation container.

3. The substance introduction unit according to claim 1, wherein the cell-impermeable member is a porous membrane having cell impermeability.

4. The substance introduction unit according to claim 1, wherein the cell-impermeable member is configured to partition the internal space of the accommodation container into an internal space where the electrode surfaces of the pair of electrodes are exposed and another internal space.

5. The substance introduction unit according to claim 4, wherein the cell-impermeable member has a cylindrical shape extending in a longitudinal direction of the accommodation container.

6. The substance introduction unit according to claim 1, wherein the cell-impermeable member is configured to partition the internal space of the accommodation container into a first internal space where an electrode surface of an anode out of the pair of electrodes is exposed and into a second internal space.

7. The substance introduction unit according to claim 1, wherein the cell-impermeable member is arranged in the accommodation container out of contact with the electrode surfaces.

8. A substance introduction device, which introduces a substance into a cell by electroporation, comprising
the substance introduction unit according to claim 1.
